(19) United States
(12) Patent Application Publication
(10) Pub. No.: US 2025/0065123 A1
(43) Pub. Date: Feb. 27, 2025

(54) INTRAVASCULAR CERVICAL SYMPATHETIC NERVE STIMULATION SYSTEM

(71) Applicant: THE TRUSTEES OF THE UNIVERSITY OF PENNSYLVANIA, Philadelphia, PA (US)

(72) Inventors: Timothy MARKMAN, Philadelphia, PA (US); Saman NAZARIAN, Bryn Mawr, PA (US)

(73) Assignee: THE TRUSTEES OF THE UNIVERSITY OF PENNSYLVANIA, Philadelphia, PA (US)

(21) Appl. No.: 18/949,254

(22) Filed: Nov. 15, 2024

Related U.S. Application Data
(63) Continuation-in-part of application No. PCT/US2023/022538, filed on May 17, 2023.
(60) Provisional application No. 63/342,788, filed on May 17, 2022.
(60) Provisional application No. 63/602,430, filed on Nov. 23, 2023.

(51) Int. Cl. A61N 1/36

(52) U.S. Cl. CPC A61N 1/36114; A61N 1/36139; A61N 1/36157; A61N 1/36171

(57) ABSTRACT

The disclosed subject matter provides techniques for treating a subject. An example method includes placing a lead in a vertebral vein of the subject, placing at least one electrode within the vertebral vein adjacent to an autonomic nerve, and modulating a cardiovascular system of the subject by supplying a predetermined amount of an electrical stimulation energy to the autonomic nerve. In non-limiting embodiments, the lead can be placed in the vertebral vein via a subclavian vein, an axillary vein, or a combination thereof. In some embodiments, the at least one electrode can be coupled to the lead.

### CROSS-REFERENCE TO RELATED APPLICATION

This application is a continuation-in-part application of International Application No. PCT/US2023/022538 filed May 17, 2023, which claims priority to U.S. Provisional Patent Application No. 63/342,788, which was filed on May 17, 2022, and claims priority to U.S. Provisional Patent Application No. 63/602,430, which was filed on Nov. 23, 2023, the entire contents of which are incorporated by reference herein.

### BACKGROUND

The autonomic nervous system (ANS) can affect the regulation of cardiovascular function. Information from a variety of inputs can be integrated into cortical centers, and regulatory functions can be transmitted through parasympathetic and sympathetic output. Maladaptive remodeling of these systems can be implicated in a variety of cardiovascular disease states, including ventricular arrhythmias, congestive heart failure, and hypertension. Disruption of homeostasis between parasympathetic and sympathetic tones can result from diverse triggers, including inflammation, glial cell activation, oxidative stress, and non-neural communication pathways. This can result in an excessive sympathetic tone with sympathoexcitatory feedback loops perpetuating imbalance.

Chronic sympathoexcitation can promote the development of myocardial fibrosis and electrical heterogeneity, promoting arrhythmias and congestive heart failure. Similarly, excessive sympathetic tone can contribute to the development and maintenance of systemic hypertension.

Certain techniques for neuromodulation have been proposed to achieve more robust sympathetic inhibition, including surgical sympathetic denervation and percutaneous stellate ganglion blockade. However, such techniques can be limited by the depth of the stimulation target and off-target effects from nearby nervous tissue.

Accordingly, there is a need for improved techniques and sampling systems to modulate a nerve system.

### SUMMARY

The disclosed subject matter provides techniques for treating a subject with an implantable electronic device for neuromodulation. An example method includes placing a lead in a vertebral vein of the subject, placing at least one electrode within the vertebral vein adjacent to an autonomic nerve, and modulating a cardiovascular system of the subject by supplying a predetermined amount of electrical stimulation energy to the autonomic nerve. In non-limiting embodiments, the lead can be placed in the vertebral vein via a subclavian vein, an axillary vein, or a combination thereof. In some embodiments, the electrode can be coupled to the lead.

In certain embodiments, the method can further include placing a pulse generator that is coupled to the lead. In non-limiting embodiments, the pulse generator can be positioned in the right ventricular apex of the subject.

In certain embodiments, the electrical stimulation energy can be continuous or titrated. In non-limiting embodiments, the electrical stimulation energy can be delivered at a magnitude of stimulation to avoid incidental stimulation of muscle.

In certain embodiments, the modulating can include inhibiting an activity of the autonomic nerve via a closed loop system function of the pulse generator when a ventricular arrhythmia is detected from the subject. In non-limiting embodiments, the modulating can include activating an activity of the autonomic nerve via a closed loop system function of the pulse generator when a thoracic impedance of the subject increases.

In certain embodiments, the electrode can be located longitudinally along a distal aspect of the lead. In non-limiting embodiments, the electrode can be positioned with a directional bias around the lead diameter. In some embodiments, the electrode can be directed toward the autonomic nerve.

In certain embodiments, the lead can include a guidewire lumen. In non-limiting embodiments, the method can further include inserting the guidewire lumen at least one centimeter into the vertebral vein and advancing the lead over the guidewire.

The disclosed subject matter provides systems for treating a subject with an implantable electronic device for neuromodulation. An example system can include a lead configured to be inserted into a vertebral vein of the subject via a subclavian vein, an axillary vein, or a combination thereof, at least one electrode coupled to the lead, and a pulse generator coupled to the lead. In non-limiting embodiments, the at least one electrode can be configured to be placed in the vertebral vein adjacent to an autonomic nerve. In some embodiments, the at least one electrode can be coupled to the lead. In non-limiting embodiments, the pulse generator can be configured to supply electrical stimulation energy to the autonomic nerve through the lead.

In certain embodiments, a frequency of the electrical stimulation energy ranges from about 0.9 Hz to about 10 kHz.

In certain embodiments, a diameter of the lead ranges from about 0.6 mm to about 2.5 mm. In non-limiting embodiments, the lead can include a tapered distal tip with a bend for positioning in the vertebral vein and ensuring contact with the autonomic nerve. In some embodiments, the lead can include up to about 20 electrodes. In non-limiting embodiments, the lead can be configured to exert a bias force to retain a distal top of the lead substantially immobile. In some embodiments, the lead can be configured to detect an electrical energy corresponding to a ventricular contraction, a thoracic impedance, or a combination thereof.

In certain embodiments, the pulse generator can be configured to detect ventricular arrhythmias based on a rate of a ventricular activity and a direction of activation. In non-limiting embodiments, the pulse generator is a battery-powered pulse generator.

The disclosed subject matter will be further described below, with reference to example embodiments shown in the drawings.

### BRIEF DESCRIPTION OF THE DRAWINGS

FIGS. 1A-1B provide a transverse CT image of the sixth cervical vertebrae (1A) and a sagittal CT image of the vertebral artery (1B) in accordance with the disclosed subject matter.

FIG. 2 provides an image showing the stimulator implanted through the left subclavian vein into the vertebral vein in accordance with the disclosed subject matter.

FIG. 3 provides an image showing an example closed loop feedback system utilizing the right ventricular lead for sensing ventricular arrhythmias and changing in thoracic impedance to modify sympathetic nerve stimulation with continuous feedback of response in accordance with the disclosed subject matter.

FIG. 4 provides anteroposterior and lateral fluoroscopy images with an electrophysiological catheter positioned in the left vertebral vein in accordance with the disclosed subject matter.

FIGS. 5A-5B provide a transverse CT image (5A) and an ultrasound image (5B) at the level of the sixth cervical vertebrae in accordance with the disclosed subject matter.

FIGS. 6A-6B provide an image and a graph showing the intravascular stimulation of the cervical sympathetic ganglia via the left vertebral veins. FIG. 6A provides a sagittal computed tomography showing the proximity of the vertebral vein and artery (arrow) to the C7 vertebral body and the anticipated region of the sympathetic ganglia (star). FIG. 6B provides a graph of acute % change in systolic blood pressure over time following stimulation in 9 patients.

FIGS. 7A-7B provide an image and a graph showing the intravascular stimulation of the cervical sympathetic ganglia via the right vertebral vein. FIG. 7A provides anteroposterior (top) and lateral (bottom) fluoroscopy images showing the electrophysiology catheter in the right vertebral vein. FIG. 7B provides a graph of acute % change in heart rate over time following right vertebral vein stimulation in five patients.

FIG. 8 provides an image and graphs showing induction of premature atrial complexes (PACs) using stimulation from the right vertebral vein.

FIG. 9 provides an image and graphs showing induction of PVCs using stimulation form the left vertebral vein.

It is to be understood that both the foregoing general description and the following detailed description are exemplary and are intended to provide further explanation of the disclosed subject matter.

### DETAILED DESCRIPTION

The disclosed subject matter provides systems and methods for treating a subject with an implantable electronic device for neuromodulation.

Unless otherwise defined, all technical and scientific terms used herein have the same meaning as commonly understood by one of ordinary skill in the art. In case of conflict, the present document, including definitions, will control. Certain methods and materials are described below, although methods and materials similar or equivalent to those described herein can be used in the practice or testing of the presently disclosed subject matter. All publications, patent applications, patents, and other references mentioned herein are incorporated by reference in their entirety. The materials, methods, and examples disclosed herein are illustrative only and not intended to be limiting.

As used in the specification and the appended claims, the singular forms “a,” “an,” and “the” include plural referents unless the context clearly dictates otherwise. Thus, for example, a reference to “a compound” includes mixtures of compounds.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, and up to 1% of a given value. Alternatively, particularly with respect to biological systems or processes, the term can mean within an order of magnitude, within 5-fold, and within 2-fold, of a value.

The term “coupled,” as used herein, includes a direct contact (e.g., mechanical contact) or an indirect coupling (e.g., a connection via one or more devices or wireless communication).

An “individual” or “subject” herein is a vertebrate, such as a human or non-human animal, for example, a mammal. Mammals include, but are not limited to, humans, primates, farm animals, sport animals, rodents, and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

The disclosed subject matter provides systems for treating a subject with an implantable electronic device for neuromodulation. An example system can include a lead, at least one electrode, and a pulse generator. The disclosed system can be configured to perform an improved or optimized selective cardiovascular sympathetic modulation through a series of tunable outputs, including amplitude, duration, and frequency of stimulation, as well as geometric steering of the current relative to the surrounding tissue.

In certain embodiments, the lead can be configured to be inserted into a vertebral vein of the subject via a subclavian vein, an axillary vein, or a combination thereof. For example, the lead can be implanted in the left vertebral vein. In some embodiments, the device can be implanted bilateral or in the right vertebral vein. In non-limiting embodiments, the lead can be permanently or temporally inserted into the venous system of the patient.

In certain embodiments, the lead placement can be optimized using a combination of fluoroscopy, ultrasound, and stimulation. For example, the lead can be implanted in a position where selective stimulation can be provided through fluoroscopy or ultrasound detection.

In certain embodiments, the lead can include platinum, iridium, silver, and/or gold-coated wires, as well as silicone, polyurethane-based insulation, or combinations thereof.

In certain embodiments, the diameter of the lead can vary depending on the target vein. For example, the diameter of the lead can range from about 0.1 mm to about 10 mm, from about 0.1 mm to about 9 mm, from about 0.1 mm to about 8 mm, from about 0.1 mm to about 7 mm, from about 0.1 mm to about 6 mm, from about 0.1 mm to about 5 mm, from about 0.1 mm to about 4 mm, from about 0.1 mm to about 3 mm, or from about 0.1 mm to about 2 mm. In non-limiting embodiments, the diameter of the lead can range from about 0.6 mm to about 2.5 mm.

In certain embodiments, the lead can be configured to be inserted into any veins of the subject. For example, the lead can include a tapered distal tip with a bend for positioning in the vertebral vein and ensuring contact with the autonomic nerve. In non-limiting embodiments, the lead can be configured to exert a bias force to retain a distal top of the lead substantially immobile.

In certain embodiments, the lead can be configured to detect an electrical energy. For example, the lead can include a sensor that can detect an electrical energy corresponding to a ventricular contraction, a thoracic impedance, or a combination thereof.

In certain embodiments, the lead can include a lead body, which further includes a guidewire lumen. In non-limiting embodiments, the lead can be inserted into a vein over the guidewire lumen. For example, a guidewire can be inserted at least one centimeter into the target vein (e.g., vertebral vein), and the lead can be advanced over the guidewire through the lumen.

In certain embodiments, the electrode can be coupled to the lead. For example, at least one electrode can be coupled to the lead. In non-limiting embodiments, the lead can include up to about 20 electrodes.

In certain embodiments, the electrode can be a stimulation electrode located at the end of a lead that can be positioned in the vertebral vein. In non-limiting embodiments, the electrode can be positioned to achieve selective capture of the cervical sympathetic nerves without capturing the surrounding muscles. In some embodiments, the electrode can be configured to be placed in the vertebral vein adjacent to an autonomic nerve.

In certain embodiments, the electrode can be positioned or added for directional stimulation and titration to ensure the long-term ability of output to selectively target the sympathetic chain. For example, the electrode can be located longitudinally along the distal aspect of the lead. In non-limiting embodiments, the electrode can be positioned with directional bias around the lead diameter. In some embodiments, the electrode closest to the target nerve can be selected for stimulation. In non-limiting embodiments, the electrode directed toward the target nerve can be selected for stimulation.

In certain embodiments, the mechanical properties of the electrode can vary depending on the target nerve and vein. The mechanical properties include size, a spacing between electrodes, and a material. In non-limiting embodiments, the electrode can include gold, silver, platinum, iridium, cobalt-chrome-molybdenum based alloys, or combinations thereof. In non-limiting embodiments, the size of the electrode can range from about 0.5 mm to 2 mm. In non-limiting embodiments, the space between electrodes can range from about 1 mm to about 10 mm.

In certain embodiments, the pulse generator can be configured to supply a predetermined amount of an electrical stimulation energy to the autonomic nerve through the lead and/or the electrode. In non-limiting embodiments, the lead and/or electrode can be coupled to the pulse generator. In some embodiments, additional lead and/or additional electrodes, which can be positioned in the right ventricular apex of the subject, can be coupled to the pulse generator. In non-limiting embodiments, the pulse generator can be a battery-powered pulse generator. In some embodiments, the pulse generator can be positioned in the right ventricular apex of the subject.

In non-limiting embodiments, the frequency of the electrical stimulation energy can range from about 0.1 HZ to about 500 kHZ, from about 0.1 HZ to about 400 kHZ, from about 0.1 HZ to about 300 kHZ, from about 0.1 HZ to about 200 kHZ, from about 0.1 HZ to about 100 kHZ, from about 0.1 HZ to about 90 kHZ, from about 0.1 Hz to about 80 kHZ, from about 0.1 Hz to about 70 KHZ, from about 0.1 Hz to about 60 kHZ, from about 0.1 Hz to about 50 kHZ, from about 0.1 Hz to about 40 kHZ, from about 0.1 Hz to about 30 kHZ, from about 0.1 Hz to about 20 KHZ, or from about 0.1 Hz to about 10 kHZ. In non-limiting embodiments, the electrical stimulation energy can be delivered at a range of frequencies from about 0.9 Hz to about 10 kHz.

In certain embodiments, the electrical stimulation frequency can vary. For example, the electrical stimulation frequency can be continuous or can be titrated based on a closed-loop system of the pulse generator.

In certain embodiments, the pulse generator can deliver the electrical stimulation energy at a predetermined magnitude of stimulation for neuromodulation. For example, the electrical stimulation energy at a magnitude of stimulation to avoid incidental stimulation of muscle can be delivered. For example, the magnitude can range from about 0.01 mA to about 30 mA.

In certain embodiments, the pulse generator can include a closed-loop feedback system/function. The pulse generator can modify the properties of the electrical stimulation energy based on the closed-loop feedback systems/functions. For example, the pulse generator can detect ventricular arrhythmias based on the rate of ventricular activity and direction of activation. When ventricular arrhythmias are detected in the subject, the pulse generator can generate electrical stimulation energy with the properties for inhibiting the autonomic nerves. For example, the properties for inhibiting the target nerves (e.g., autonomic nerves) can include a high frequency (e.g., about 100 hz-about 10 khz), low frequency (e.g., about 0.9 Hz-about 5 Hz), an amplitude (about 0.01 mA-about 30 mA), a pulse width (e.g., about 1 microsecond-about 1 millisecond), or combinations thereof. In non-limiting embodiments, when the lead senses the increased thoracic impedance, the pulse generator can generate an electrical stimulation energy with properties for activating the autonomic nerves based on the closed loop feedback system/function. For example, the properties for activating the target nerve (e.g., autonomic nerves) can include a mid-range frequency (e.g., about 6 Hz-to about 99 Hz), an amplitude (e.g., about 0.01 mA-about 30 mA), a pulse width (e.g., about 1 microsecond-about 1 millisecond), or combinations thereof.

The disclosed subject matter also provides methods of treating a subject, e.g., with the disclosed system. An example method includes placing a lead in a vertebral vein of the subject, placing at least one electrode within the vertebral vein adjacent to an autonomic nerve, and modulating the cardiovascular system of the subject by supplying a predetermined amount of electrical stimulation energy to the autonomic nerve.

In certain embodiments, the lead can be placed in the vertebral vein via a subclavian vein, an axillary vein, or a combination thereof. In non-limiting embodiments, the electrode can be located longitudinally along a distal aspect of the lead. In non-limiting embodiments, the electrode can be positioned with a directional bias around the lead diameter. In some embodiments, the electrode can be directed toward the autonomic nerve.

In certain embodiments, the vertebral vein lead can be configured to measure and/or record the activity of the target nerve (e.g., sympathetic nerves). In non-limiting embodiments, the disclosed modulation of the outputs can be performed based on the detected activity of the target nerve (e.g., sympathetic nerves) in a closed-loop system.

In certain embodiments, the method can further include placing a pulse generator. For example, the pulse generator can be positioned in the right ventricular apex of the subject. In non-limiting embodiments, the pulse generator can be positioned outside of the subject's body.

In certain embodiments, modulating the cardiovascular system of the subject can include inhibiting an activity of the autonomic nerve via the closed loop system function of the pulse generator when a ventricular arrhythmia is detected from the subject. In non-limiting embodiments, modulating the cardiovascular system of the subject can include activating an activity of the autonomic nerve via a closed-loop system function of the pulse generator when a thoracic impedance of the subject increases.

In certain embodiments, the method can further include inserting the guidewire lumen at least one centimeter into the vertebral vein and advancing the lead over the guidewire.

In certain embodiments, the method can further include modifying the electrical stimulation energy. For example, the properties of the electrical stimulation energy can be modified to have properties that can modulate the target nerves via a vein for controlling cardiovascular sympathetic tone. The electrical stimulation energy can have properties that can be enough to modulate the target nerves without stimulating non-target tissues (e.g., muscle). For example, the electrical stimulation energy can include a stimulation amplitude (e.g., about 0.01 mA-about 30 mA), a pulse width (e.g., about 1 microsecond-about 1 millisecond), or a combination thereof.

The disclosed subject matter can be used to control an excessive sympathetic tone, which can cause cardiovascular diseases (e.g., ventricular arrhythmias, congestive heart failure, and hypertension) via veins. The disclosed subject matter can perform control of the sympathetic tone and the neuromodulation through a vertebral vein of a subject. In non-limiting embodiments, the disclosed subject can also be used for the neuromodulation of the cervical sympathetic ganglion with an assay of biomarkers to allow for improvement or optimization of “on-target” stimulation.

In certain embodiments, the disclosed device can access a target area of the body through the vasculature with catheters. The disclosed device can access the area, which can be more challenging with surgical techniques and more associated with complexity/risk, through the vasculature with catheters. For example, certain pacemakers and defibrillators can be placed directly on the heart but this is done a less invasive and save manner through the vasculature. Another example is phrenic nerve stimulation for central sleep apnea. Direct diaphragmatic stimulators can be challenging to adopt because of surgical issues with implants but transvenous stimulators are safely placed. Another example is the BaroStim stimulator placed directly on the carotid; certain existing techniques use vascular surgery, which has limited its adoption due to surgical dissection through “high risk real-estate” of the neck. The disclosed subject matter can avoid such risks by accessing the target area through the vasculature with catheters.

In certain embodiments, the disclosed subject matter can be used for providing excitatory stimulation effects and/or inhibitory effects on the target area. For example, certain frequencies (e.g., about 1 Hz-about 100 Hz or about 1 Hz-about 1000 Hz) can excite the target tissue (e.g., heart).

In certain embodiments, the disclosed subject matter can be used for reducing or decreasing the stimulation of the target tissue/area. For example, certain frequencies (e.g., below about 1 Hz or above 1 kHz) can have inhibitory effects on the target tissue/area.

In certain embodiments, the parameters of the disclosed devices (e.g., frequency, amplitude, treatment duration, or combinations thereof) can be adjusted depending on target tissues, desired therapeutic effects, or conditions of a subject. For example, for clinical conditions where sympathetic stimulation (e.g., vasovagal syncope or orthostasis) or where sympathetic inhibition (e.g., ventricular arrhythmias, hypertension, angina, inappropriate sinus tachycardia or heart failure) is desired, a user can use different frequencies, amplitude, treatment time, or combinations thereof.

In certain embodiments, the disclosed device can provide a wide therapeutic window where hemodynamic effects and no off-target effects are detected. This window can be larger than for direct stimulation because the disclosed device can access closer to the target nerve and not going through the other tissue. In non-limiting embodiments, the disclosed device can provide the disclosed clinical effects without evidence of off-target stimulation with outputs ranging from about 2 to about 30 mA in certain subjects. In non-limiting embodiments, the disclosed device can include a directional stimulation catheter that can increase the therapeutic window as stimulation energy can be pointed at the target nerve directly.

In certain embodiments, the disclosed device can be used for the long term (e.g., permanent implanted device). For example, the disclosed device can be implanted for a subject with a target condition for long-term treatments (e.g., vasovagal syncope, orthostatic syncope, hypertension, heart failure, cardiac arrhythmias, coronary artery disease, or combinations thereof).

In certain embodiments, the disclosed device can be used for a short term (e.g., during an acute hospitalization for up to about 1 week). For example, the disclosed device can be used for a subject with a target condition for short-term treatments (e.g., acute presentations of ventricular arrhythmia storm for acute interruption of sympathetic tone or potentially acute increase in blood pressure and heart rate in other select conditions).

In certain embodiments, the disclosed device can be used for treating or preventing vasovagal or orthostatic syncope. The disclosed techniques can simultaneously increase heart rate (HR) and blood pressure (BP). For example, the disclosed devices and techniques can be used to treat conditions that are marked by transient low heart rate (HR) and blood pressure (BP). In non-limiting embodiments, the disclosed device can simultaneously increase HR and BP with one treatment.

In certain embodiments, the disclosed device can provide inhibitory effects by decreasing BP and can be a titratable implant to lower BP. In certain embodiments, the disclosed device can include a battery that can be physically replaced. In non-limiting embodiments, transcutaneous charging can be used.

In certain embodiments, the presently disclosed subject matter provides a system for guiding a cardiac ablation procedure. In certain embodiments, the system can induce an arrhythmia.

In certain embodiments, the presently disclosed subject matter provides a device that can induce an arrhythmia.

In certain embodiments, the arrhythmia can include premature atrial complexes, premature ventricular complexes, supraventricular tachycardia, and ventricular tachycardia. In certain embodiments, the arrhythmia is transiently induced. In certain embodiments, the induced arrhythmia allows for the approximation of the arrhythmia site of origin. In certain embodiments, the origin site of the induced arrhythmia is located through pace mapping. In certain embodiments, the induced arrhythmia can be used to guide an ablation procedure. In certain embodiments, the ablation procedure is a catheter ablation.

### EXAMPLES

### Example 1: Development of a Transvenous Implantable Electronic Device for Neuromodulation of the Cervical Sympathetic Ganglia

The disclosed subject matter provides a transvenous implantable electronic stimulator for neuromodulation of the cervical sympathetic ganglia. Targeting these ganglia with an implantable stimulator can be a therapeutic approach that is durable while maintaining reversibility and offering innovative treatment for multiple cardiovascular conditions.

Direct electrical stimulation of peripheral nerves can result in decreased excitability with the resultant increase in the stimulation threshold and slowing of conduction velocities. Low-frequency stimulation (˜1 Hz) can achieve inhibition through long-term depression with effects that can outlast the duration of stimulation.

While low-frequency stimulation can achieve inhibition of peripheral nerves, effects can vary as stimulation frequency in the 10-100 Hz range results in excitation, and higher frequencies (>100 Hz) result in inhibition. When nerves are stimulated at a rate >100 Hz, neurotransmitters can be depleted, and a functional block is achieved, while stimulation with kilohertz frequency alternating current results in a direct conduction block. The inhibition achieved with higher frequency stimulation can be valuable as it is immediate and absolute while remaining non-destructive, reversible, and titratable.

Although certain techniques (e.g., transcutaneous magnetic stimulation) can be used for sympathetic neuromodulation of cardiovascular disease, they can be limited by an inability to apply kilohertz frequency stimulation due to coil heating. Furthermore, due to anatomic variation in the depth from skin to the cervical sympathetic chain, such techniques can be limited by an inherent trade-off between focality of the targeted region and the depth of stimulation. With excessive target depth relative to the skin surface, there can be an unavoidable risk of off-target stimulation, which can include both stimulations of nearby nerves and as well as potentially painful muscle stimulation.

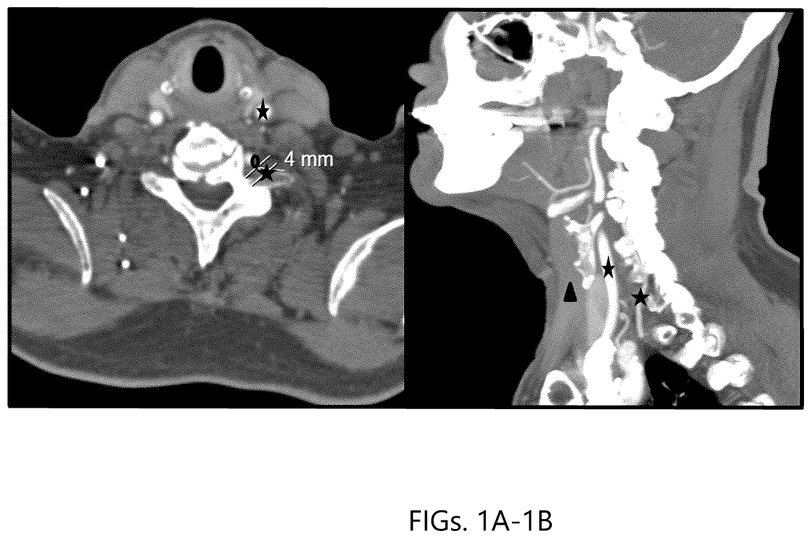

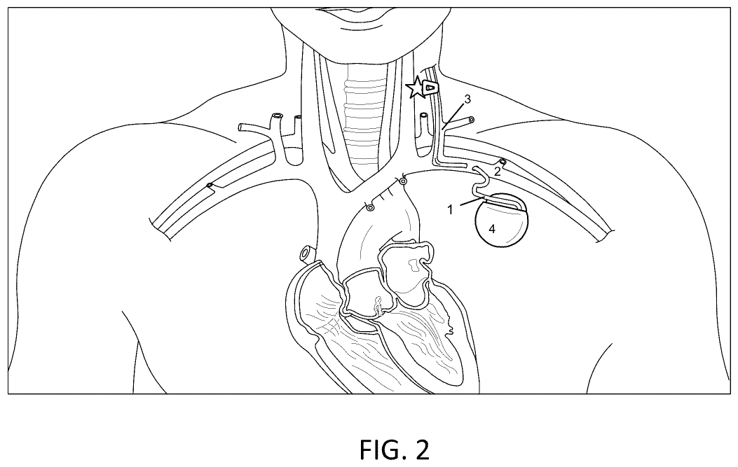

The cervical sympathetic chain is located posteromedial to the carotid artery & anterior to the longus coli muscles, and stellate ganglion is located anterior to the sixth & seventh cervical vertebrae. Although the depth relative to the skin can be variable based on body habitus, there can be a consistent relationship between the stellate and the vertebrae as well as the major vessels of the head and neck. The vertebral vein can drain blood from the cervical spine and travels from the skull base in the transverse foramina of the cervical vertebrae before exiting at the level of the sixth cervical vertebrae and coursing laterally and anteriorly to drain into the brachiocephalic vein near its origin. Therefore, the vertebral veins can consistently cross over the longus coli muscle and the cervical sympathetic chain at the location of the stellate ganglion (FIG. 1). FIG. 1A shows an example of transverse CT images of sixth cervical vertebrae. FIG. 1B shows an example sagittal CT image of the vertebral artery, which can be paired to the vertebral vein, entering the transverse foramina at the level of the stellate ganglion. The vertebral vein (circle) is seen <5 mm anterior to the stellate ganglion (star). Carotid arteries (black star) and sternocleidomastoid muscles (triangle) are located anteriorly to these structures.

As opposed to surgical sympathetic denervation, the disclosed implantable stimulation devices can be non-destructive and reversible, allowing tunable outputs based induce optimal therapeutic effects while minimizing both on-target and off-target adverse effects. Unlike other non-destructive neuromodulation strategies (e.g., stellate ganglion block or TcMS), which need to be repeated to achieve continued therapeutic effects, the disclosed implantable stimulator can offer a durable treatment solution.

As shown in FIG. 2, the disclosed stimulator (1) can be implanted through the left subclavian vein (2) into the vertebral vein (3), where the current is directed to stimulate the cervical sympathetic ganglion (star). The generator can be positioned in a subcutaneous pocket (4).

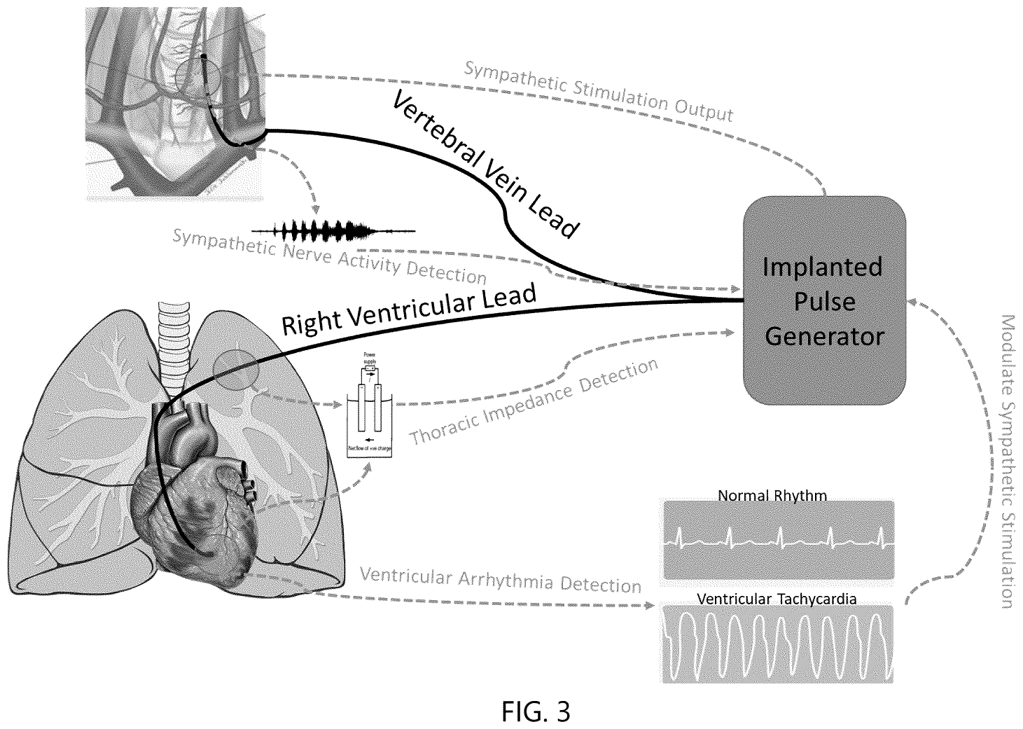

Furthermore, the disclosed implantable devices can be used for closed-loop applications, such as a system with tunable output based on the constant readout of biomarkers, which can be routinely measured by implantable electronic devices (e.g., ventricular arrhythmia burden, thoracic impedance, or systemic vascular resistance, FIG. 3).

FIG. 3 shows an example closed loop feedback system utilizing right ventricular lead for sensing ventricular arrhythmias and changes in thoracic impedance to modify sympathetic nerve stimulation with continuous feedback of response. The disclosed subject matter provides improved tunability and avoids a more invasive surgery with associated risk and adverse effects.

The disclosed stimulator can be inserted percutaneously (e.g., in a method similar to that safely used by standard cardiac implantable electronic devices). The stimulator can include a generator to be positioned in a subcutaneous pocket and a stimulation electrode at the end of a lead that can be positioned in the vertebral vein (FIG. 2). Permanent lead implantation in the venous system can be safe and routinely performed for implantable electronic devices. Due to the predominant innervation of the ventricle from the left sympathetic chain and the preponderance of clinical evidence from left stellate ganglion inhibition, initial stimulators can be implanted in the left vertebral vein. The bilateral and right-sided implants are also evaluated. This stimulator can be capable of optimized selective cardiovascular sympathetic modulation through a series of tunable outputs, including amplitude, duration, and frequency of stimulation, as well as geometric steering of the current relative to the surrounding tissue.

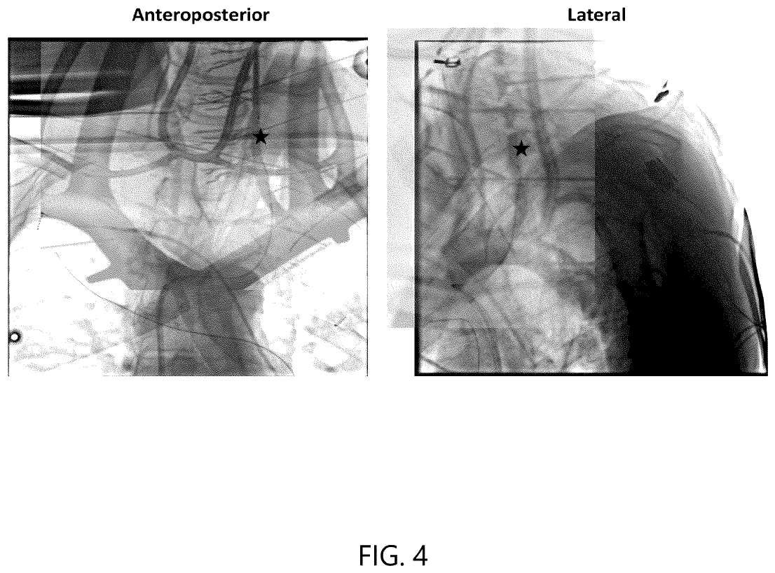

The disclosed stimulator can target the cervical sympathetic chain by means of pulsed electrical output. The disclosed system can be designed to improve or maximize the tunability of sympathetic modulation both at the time of implantation and for chronic titration/optimization. At the time of implantation, the lead positioning can be performed to achieve the optimal degree of site-specific stimulation. Chronically, the system can be modifiable with regard to the geometric direction of the output in addition to the duration, amplitude, frequency, and pulse width of stimulation. These parameters can be tuned both to achieve optimal sympathetic inhibition but also to avoid off-target effects. Potential off-target effects can include those resulting from stimulation of nearby nervous structures (i.e., carotid sinus, vagus nerve, spinal cord) and capture of surrounding muscles (See FIG. 1). Although the anatomic proximity of these structures to the sympathetic chain can limit the ability to achieve selective stimulation transcutaneously, the vagus nerve can be anterior, and the carotid sinus can be both caudal (level of fourth cervical vertebrae) and anterior to the sympathetic chain and vertebral vein. This separation can allow for selective stimulation with a generous safety margin, as shown in the human stimulation. At the time of implantation, lead placement can be optimized using a combination of fluoroscopy, ultrasound, and/or stimulation. An electrode can be positioned to achieve selective capture of the cervical sympathetic nerves without the capture of the surrounding muscles (FIG. 4). FIG. 4 shows the anteroposterior and lateral fluoroscopy with an electrophysiological catheter positioned in the left vertebral vein. Anatomic cartoon protection overlying the fluoroscopic images. The level of stimulation with the physiological effect observed is denoted with a black star.

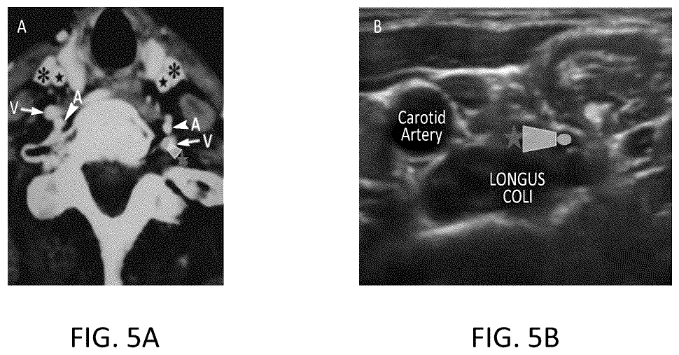

The lead can therefore be implanted in a position where selective stimulation is known to be achievable. The addition of an electrode with directional stimulation, as proposed, can allow for additional titration to ensure the long-term ability of output to selectively target the sympathetic chain (FIG. 5).

FIG. 5 shows a transverse CT (5A) and ultrasound (5B) at the level of the sixth cervical vertebrae. A stimulation electrode can be positioned in the vertebral vein (oval) with an electrical output (trapezoid) directed toward the stellate ganglion (star) away from the sternocleidomastoideollowing lead positioning and current steering, the stimulation can be additionally tuned based on output amplitude, frequency, and pulse width. The amplitude and pulse width of stimulation can affect the degree of nerve activity suppression with larger current output resulting in greater suppressive effects. The disclosed system can provide the amplitude and duration-dependent hemodynamic effects with excitatory sympathetic stimulation protocols from the vertebral vein in three humans.

The stimulation frequency of the disclosed device can be fully tunable from low frequency (e.g., <1 hz) to high frequency (e.g., >1 kilohertz) to achieve the optimal degree of sympathetic inhibition for an individual patient.

The disclosed device can allow for a range of stimulation protocols, including chronic (“always-on”) stimulation, intermittent (e.g., daily stimulation sessions), or “on-demand” (e.g., based on closed-loop algorithms that can activate inhibitor protocols based on ventricular ectopy as a marker of arrhythmogenesis, thoracic impedance as a marker of congestive heart failure, or systemic vascular resistance as a marker of blood pressure). For example, an additional lead can be connected to the generator and positioned in the right ventricle that can sense ventricular activation to detect arrhythmias or thoracic impedance as a marker of heart failure in order to titrate stimulation output with continuous monitoring (FIG. 3).

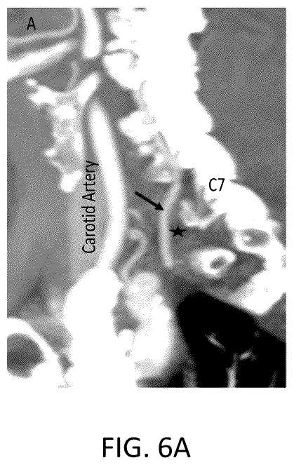

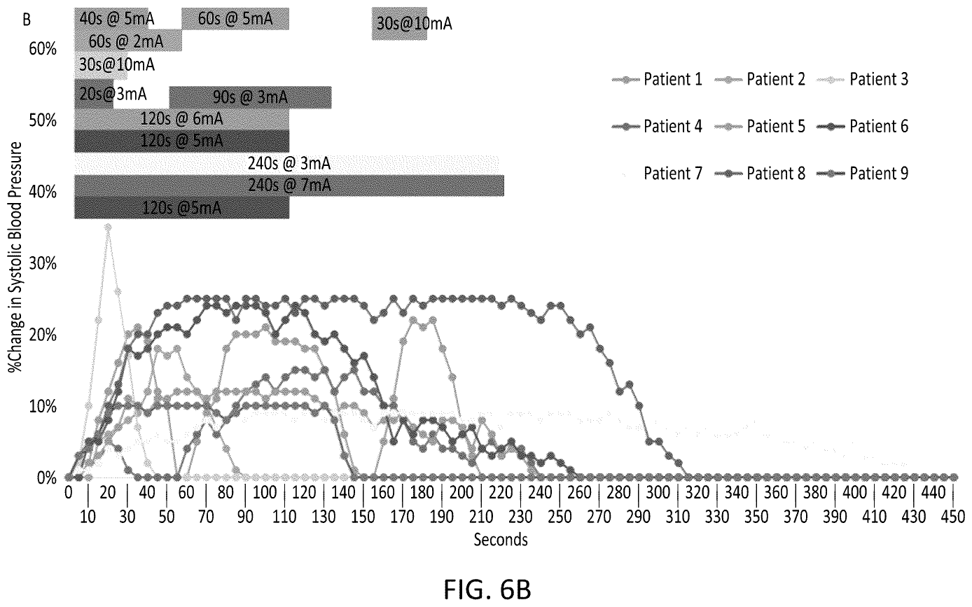

Hemodynamic Parameters: Acute inhibition of the sympathetic chain can be titrated based on changes in hemodynamic parameters: heart rate, blood pressure, left ventricular contractility (dP/dT), and/or left ventricular volume load (end-diastolic pressure). These parameters can be reliably and reproducibly measured and modified by stimulation of the cervical sympathetic nerves (FIG. 6A). Clinical results demonstrating the ability to titrate blood pressure in patients based on stimulation amplitude to achieve target blood pressures with 10 mmHg mean arterial are shown (FIG. 6B).

Similarly, the magnitude of the effect is observed: a 20% decrease in systolic blood pressure, a 10% reduction in left ventricular dP/dT, and a 30% reduction in left ventricular end-diastolic pressure. These are both markers of stable and tunable sympathetic stimulation and also valuable endpoints for the treatment of hypertension as currently available interventions for hypertension frequently produce more modest blood pressure reduction.

Biochemical Parameters: Real-time assessment of these hemodynamic parameters as well as the measurement of circulating and local myocardial levels of norepinephrine and neuropeptide Y, both of which are markers of cardiac sympathetic activity and are modified by sympathetic inhibition, can be performed. Stimulation of the stellate ganglion can reliably produce a >100% increase in both norepinephrine and neuropeptide Y levels within 15 minutes, and the 50% reduction in both biomarkers can be achieved consistent with successful inhibition.

Electrophysiological: The disclosed stimulator can provide for evaluation of electrophysiologic biomarkers that can be affected by sympathetic inhibition but can be individually evaluated with an easily titrated simulation device. For example, skin sympathetic nerve activity and the galvanic skin response can be a marker of cardiac sympathetic tone. These can be evaluated to identify the potential for use as biomarkers for the acute and chronic titration of sympathetic inhibition. This can include surface electrocardiogrameters (e.g., QTc interval) and invasive assessment of ventricular conduction velocities and refractory periods.

Off-Target Effects: The off-target and adverse effects can be monitored closely with additional biomarkers to ensure selective and appropriate sympathetic inhibition. Inadvertent inhibition of the vagus nerve can be anticipated to produce paradoxical tachycardia in addition to non-cardiac manifestations such as hypoxia, gastrointestinal distress, urinary retention, vocal cord dysfunction and neck pain, all of which can be monitored clinically and with standard assays (e.g., pulse oximetry, bladder scanning, real-time patient symptom assessment). There is also a risk of seizure induction with vagal inhibition, and this can be monitored clinically, and initial human experiments will exclude patients with a history of epilepsy. There can also be adverse effects of “on-target” stimulation that need to be monitored, and stimulation can be titrated to avoid these effects. This includes hypotension, bradycardia, or Horner's syndrome, which can result from disruption of cervical sympathetic nerves. Hemodynamics can be carefully monitored to titrate output. Inhibition of sympathetic tone without producing Horner's syndrome can be achievable, and unlike destructive techniques, which can result in permanent damage, this approach can be titrated (both direction and intensity of stimulation) to ensure a safety margin without producing this adverse effect.

Cardiovascular disease remains the leading cause of death in the United States, with an increasing prevalence of ventricular arrhythmias, congestive heart failure, and hypertension. The global economic burden of congestive heart failure alone exceeds $100 billion annually, and these costs continue to rise. The disclosed device can be used for the treatment of the following three indications because of both their burden on morbidity and mortality as well as the inherent connection to the autonomic nervous system. The disclosed subject matter also provides findings from a tunable implantable sympathetic inhibition device for new insights into the interaction between autonomic and cardiovascular systems and novel therapeutic strategies.

Ventricular Arrhythmias: Every year, an estimated 300,000 Americans experience sudden cardiac death, which is frequently attributable to ventricular arrhythmias. Although implantable cardioverter-defibrillators can be the mainstay of treatment for the prevention of sudden cardiac death, these devices abort sudden death but do not prevent ventricular arrhythmias. Ventricular arrhythmias can be closely linked to excessive sympathetic tone, and although neuromodulation therapies are frequently employed in this population, they can be limited by the need for destructive or repeat procedures and the inability to tune sympathetic inhibition relative to variable arrhythmia burden. Patients can benefit from a closed-loop system in which ventricular ectopy is detected as a marker of arrhythmogenicity, resulting in increased sympathetic inhibition.

Congestive Heart Failure: The lifetime risk of heart failure in the United States is 20%, with >650,000 incidence cases annually and a continually growing prevalence of this chronic condition. Despite improvements in pharmacotherapy, it remains progressive, resulting in recurrent hospitalizations and 2% of the total healthcare expenditures in the United States. Given the dynamic nature of heart failure, there can be substantial value in a closed-loop system that can titrate autonomic tone based on real-time feedback of thoracic impedance.

Hypertension: More than a quarter of the world's adult population is estimated to have hypertension, which is a major contributor to cardiovascular and cerebrovascular morbidity and mortality. The annual cost of hypertension-related hospitalizations exceeds $100 billion, and that number continues to rise. Effective pharmacologic treatments for hypertension are limited by poor compliance in part due to “off-target” side effects and the inherent challenges of frequent and complex medication dosing regimens. Furthermore, blood pressure is dynamic, and control of hypertension is often limited by period hypotensive episodes. By limiting the reliance on the patient and by capitalizing on closed-loop algorithms that titrates sympathetic inhibition based on continually measured markers such as systemic vascular resistance, the disclosed device can benefit millions of patients with uncontrolled hypertension.

Maladaptive remodeling of the autonomic nervous system can affect numerous cardiovascular diseases, including ventricular arrhythmias, congestive heart failure, and hypertension. The excessive sympathetic tone in these disease states can present an important opportunity for therapeutic neuromodulation. The disclosed subject matter can utilize the vertebral vein, a unique avenue for an implantable stimulator designed to selectively inhibit cardiovascular sympathetic tone. The disclosed subject matter provides a reliable means of cannulating this vein, which can demonstrate a consistent doseresponse to sympathetic stimulation. The disclosed implantable electronic stimulation device can be used for the neuromodulation of the cervical sympathetic ganglion with various assays of biomarkers to allow for the optimization of “on-target” stimulation.

### Example 2: Intravascular Cervical Sympathetic Nerve Stimulation

In this example, a novel intravascular technique for neuromodulation of the cervical sympathetic ganglia is presented.

The cervical sympathetic ganglia are located posteromedial to the carotid artery and anterior to the Longus coli muscles at the level of the sixth & seventh cervical vertebrae. Although the depth relative to the skin can be variable based on body habitus, limiting transcutaneous approaches, there is a consistent relationship between the ganglia and the vertebrae as well as the major vessels. For example, the vertebral veins drain blood from the cervical spine and travel bilaterally from the skull base in the transverse foramina of the cervical vertebrae before exiting at the level of the sixth cervical vertebrae and coursing laterally and anteriorly to drain into the brachiocephalic veins. Therefore, the vertebral veins consistently cross over the Longus coli muscle and the cervical sympathetic chain (FIG. 6A). This anatomic relationship presents an opportunity for an intravascular approach to sympathetic stimulation.

In 13 patients undergoing catheter ablation, left vertebral vein cannulation from a femoral venous approach was attempted. In app patients, blood pressure was continuously monitored with a radial arterial catheter and continuous telemetry. In the first 3 patients, the left vertebral vein was cannulated with a standard 0.032″ J-tipped guidewire. The position of the wire was confirmed based on fluoroscopy with a posterior position coursing toward the vertebrae. In 9 subsequent patients, the left vertebral vein was cannulated with a 2 Fr octapolar electrophysiologic catheter (1.3-1.5 mm electrodes, 5 mm electrode spacing). In one patient with a left-sided dual-chamber ICD, the vertebral vein could not be cannulated and was presumed to be occluded due to the presence of indwelling leads. With pacing at 20 Hz and 2-10 mA for up to 240 seconds, reproducible hemodynamic changes were observed (FIG. 6B).

Each patient had a transient increase in blood pressure, and an increase in heart rate was noted in two patients. Stimulation was only noted to have a hemodynamic effect from a single bipolar electrode for each patient, suggesting this was the particular level where the vein crossed adjacent to the sympathetic nerves. Following stimulation, blood pressure and heart rate returned to baseline, with the duration of effects noted to be proportional to the duration of stimulation. Electrical stimulation of peripheral nerves can modify excitability with resultant changes in the stimulation threshold and conduction velocities.

This example shows direct cervical sympathetic stimulation through the vertebral vein. The ability to undertake a transvenous approach can provide advantages, including the ability to stimulate synchronous to catheter ablation. No complications related to cannulation or stimulation were shown when performed by experienced operators.

Table 1 shows the physiologic effects of intravascular stimulation of the cervical sympathetic ganglia via the left vertebral vein. Maximal percent changes in baseline heart rate, systolic blood pressure, and diastolic blood pressure are reported for each patient who underwent stimulation. Latency of peak effect from initiation of stimulation and time until normalization of hemodynamics after the termination of stimulation is also shown (bpm: beats per minute; mA: milliamps).

TABLE 1
Physiologic effects of intravascular stimulation of the cervical sympathetic ganglia via the left vertebral vein.

| | | | Maximal | Maximal | Maximal | | Time to |
|---|---|---|---|---|---|---|---|
| | Maximal | | Change in | Change in | Change in | Time to | normalization of |
| | Stimulation | Stimulation | Heart | Systolic Blood | Diastolic Blood | peak | hemodynamic |
| Patient | Amplitude | Duration | Rate | Pressure | Pressure | effect | parameters |
| # | (mA) | (seconds) | (bpm) | (mmHg) | (mmHg) | (seconds) | (seconds) |
| 1 | 10 | 30 | <5 | 43 | 13 | 26 | 23 |
| 2 | 2 | 60 | <5 | 18 | 2 | 42 | 28 |
| 3 | 10 | 30 | <5 | 24 | 10 | 20 | 16 |
| 4 | 3 | 90 | <5 | 19 | 2 | 60 | 84 |
| 5 | 6 | 120 | <5 | 11 | 3 | 54 | 118 |
| 6 | 5 | 120 | 23 | 26 | 9 | 70 | 136 |
| 7 | 3 | 240 | <5 | 14 | 1 | 87 | 201 |
| 8 | 7 | 240 | 17 | 22 | 8 | 56 | 76 |
| 9 | 5 | 120 | <5 | 8 | 7 | 25 | 21 |

### Example 3: Physiologic Effects of Right-Sided Intravascular Cervical Sympathetic Nerve Stimulation

The maladaptive remodeling of the sympathetic nervous system can be implicated in the pathogenesis of cardiovascular disease, including ventricular arrhythmias. Certain strategies for neuromodulation can have substantial limitations (e.g., none are easily modified to enhance sympathetic tone when desired or performed synchronously to invasive electrophysiologic procedures).

The disclosed device can provide intravascular stimulation via the left vertebral venous system for neuromodulation of the cervical sympathetic ganglia. The cervical sympathetic ganglia are located posteromedial to the carotid artery and anterior to the Longus coli muscles at the level of the sixth & seventh cervical vertebrae. There is a consistent anatomic relationship between the ganglia and the vertebrae, as well as the major vessels of the head and neck. For example, the vertebral venous system drains blood from the cervical spine, travels bilaterally as plexuses from the skull base in the transverse foramina of the cervical vertebrae before exiting at approximately level of the seventh cervical vertebrae and coursing laterally and anteriorly in larger vessels, ultimately drain into the brachiocephalic veins. Therefore, the vertebral veins consistently cross over the Longus coli muscle and the cervical sympathetic chain at the location of the cervical sympathetic ganglia. This anatomic relationship can present an opportunity for an intravascular approach to sympathetic stimulation. The disclosed subject matter demonstrated that stimulation of the left cervical ganglia with this approach resulted in reproducible hemodynamic effects with a relatively selective increase in blood pressure that was transient and with duration and magnitude of effect proportional to duration and amplitude of stimulation.

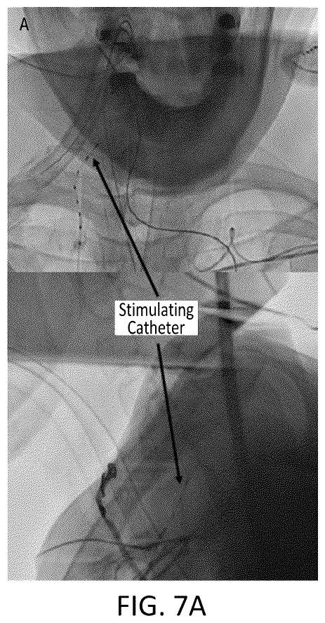

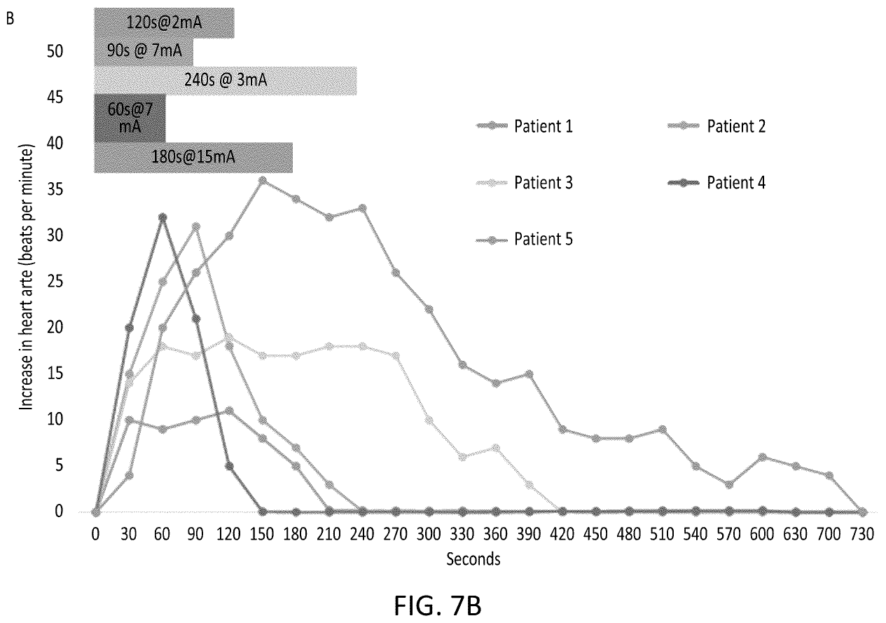

In 5 patients undergoing catheter ablation for atrial fibrillation under general anesthesia, the right vertebral vein was cannulated from a femoral venous approach (FIG. 7A). Blood pressure was continuously monitored with a radial arterial catheter, and all patients were monitored on continuous telemetry. Cannulation was performed with a 2 Fr octapolar electrophysiologic catheter (1.3-1.5 mm electrodes, 5 mm electrode spacing), which was advanced to the cervical vertebrae. With pacing at 20 Hz and 2-15 mA for up to 4 minutes, reproducible hemodynamic changes were observed (FIG. 7B).

Each patient had a transient increase in heart rate (range 11-32 bpm), and a significant increase in blood pressure was noted in three patients (10, 17, and 32 mmHg, respectively). Following stimulation, both blood pressure and heart rate returned to baseline levels. One patient, who was under general anesthesia, was noted to cough during stimulation at 7 mA. When the output was decreased to 5 mA, no additional coughing was noted. There were no complications, although it is important to note that this technique was performed by experienced operators in an investigational setting. Routine use of vertebral venous cannulation and stimulation is not encouraged at this time.

A transvenous approach to direct sympathetic nerve stimulation can produce both acute and chronic effects on nerve activity. The acute effects of right-sided cervical sympathetic ganglia are predominately on heart rate, consistent with principal atrial innervation. The disclosed subject matter shows direct cervical sympathetic stimulation through the right vertebral venous system has important potential applications, especially for use at the time of invasive electrophysiologic procedures, where selective modulation of atrial autonomic innervation can be valuable.

### Example 4: Induction of Arrhythmias Using Sympathetic Stimulation Via the Vertebral Vessels

Ablation of arrhythmias such as premature atrial complexes (PACs), premature ventricular complexes (PVCs), supraventricular tachycardia, or ventricular tachycardia can rely on or benefit from the induction of the arrhythmia. In cases where the arrhythmia cannot easily be induced, the efficacy or efficiency of the procedure can be limited. In some cases, when arrhythmias cannot be observed, the procedure must be aborted entirely. The disclosed subject matter provides improvements to existing methods of arrythmia ablation by using sympathetic stimulation via the vertebral vessels to induce clinical arrhythmias. The reliable induction of the arrythmia allows for accurate mapping of the arrythmia and for the guided ablation.

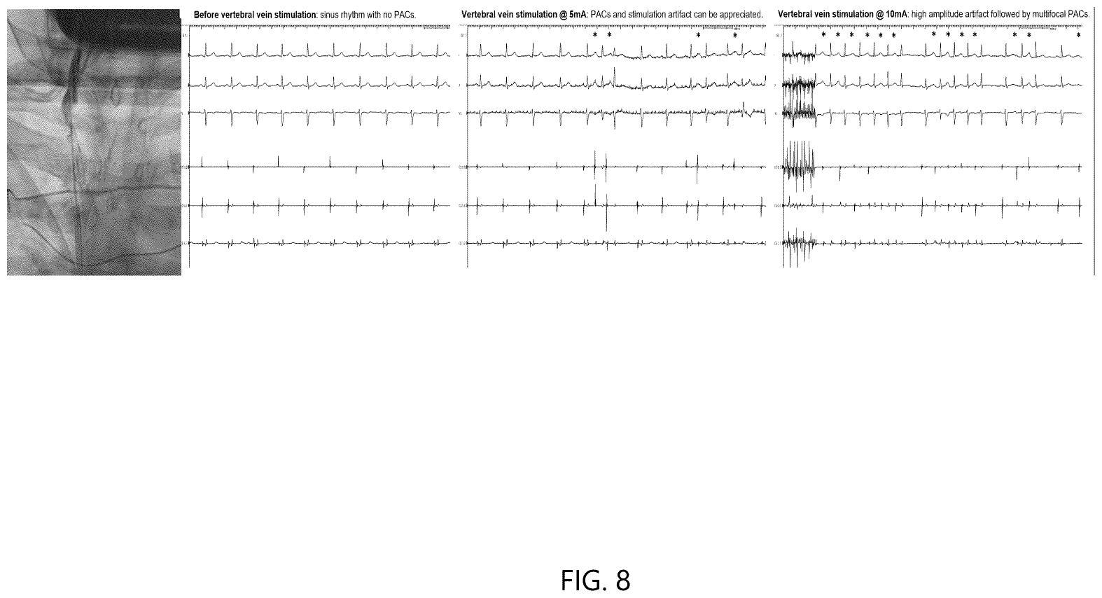

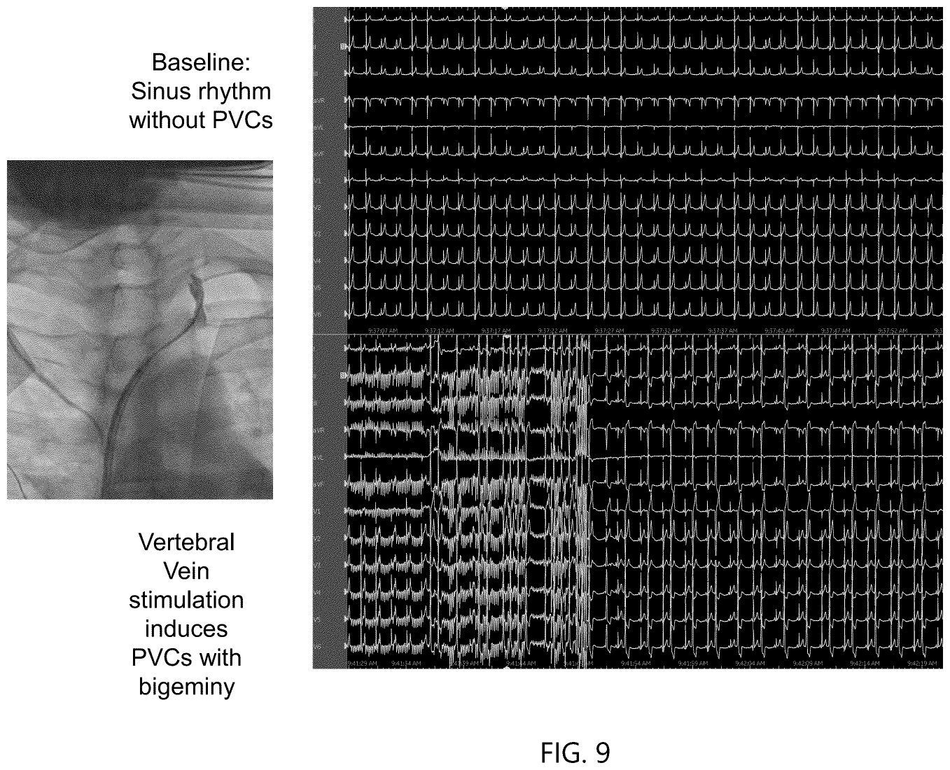

Premature atrial complexes (PACs) were reliably induced using stimulation from the right vertebral vein as previously described with 20 Hz pacing at 2-20 mA for up to 15 minutes (FIG. 8), while premature ventricular complexes (PVCs) were induced through stimulation from the left vertebral vein (FIG. 9). The disclosed subject matter shows that arrhythmias can be reliably induced in both the upper atrial and lower ventricular chambers. Induction of premature atrial or premature ventricular complexes allows for approximation of the arrhythmia site of origin and subsequent pace-mapping to guide ablation.

Induction of frequent ectopy allows for activation mapping to guide ablation. Induction of supraventricular tachycardia or ventricular tachycardia allows for characterization of the arrhythmia (including pathological mechanisms), associated risk of sudden death, and site of origin. Inducing the arrythmia allows for its mapping and subsequent ablation.

Repeat sympathetic nerve stimulation using the disclosed subject matter following the ablation procedure can be used as a marker of success or failure of the ablation.

In additional, vertebral vein stimulation can be performed to assess the propensity of a patient to develop arrhythmias which can help to determine management including identifying, titrating, or optimizing various medications, as well as implanting a cardioverter defibrillator for the prevention of sudden death.

All patents, patent applications, publications, product descriptions, and protocols, cited in this specification are hereby incorporated by reference in their entireties. In case of a conflict in terminology, the present disclosure controls.

While it will become apparent that the subject matter herein described is well calculated to achieve the benefits and advantages set forth above, the presently disclosed subject matter is not to be limited in scope by the specific embodiments described herein. It will be appreciated that the disclosed subject matter is susceptible to modification, variation, and change without departing from the spirit thereof. Those skilled in the art will recognize or be able to ascertain using no more than routine experimentation, many equivalents to the specific embodiments described herein. Such equivalents are intended to be encompassed by the following claims.

What is claimed is:

1. A method of treating a subject with an electronic device for neuromodulation, comprising:
placing a lead in a vertebral vein of the subject, wherein the lead is placed in the vertebral vein via a subclavian vein, an axillary vein, or a combination thereof;
placing at least one electrode within the vertebral vein adjacent to an autonomic nerve, wherein the at least one electrode is coupled to the lead; and
modulating a cardiovascular system of the subject by supplying a predetermined amount of an electrical stimulation energy to the autonomic nerve.

2. The method of claim 1, wherein the method comprises inducing an arrhythmia.

3. The method of claim 2, wherein the method further comprises identifying a site of origin based on the arrhythmia.

4. The method of claim 3, further comprising guiding an ablation based on the arrhythmia.

5. The method of claim 1, wherein the electronic device is implantable.

6. The method of claim 5, further comprising placing a pulse generator that is coupled to the lead.

7. The method of claim 5, wherein the electrical stimulation energy is continuous or titrated.

8. The method of claim 5, wherein the electrical stimulation energy is delivered at a magnitude of stimulation to avoid incidental stimulation of muscle.

9. The method of claim 6, wherein the modulating the cardiovascular system of the subject comprises inhibiting an activity of the autonomic nerve via a closed loop system function of the pulse generator when a ventricular arrhythmia is detected from the subject.

10. The method of claim 6, wherein the modulating the cardiovascular system of the subject comprises activating an activity of the autonomic nerve via a closed loop system function of the pulse generator when a thoracic impedance of the subject increases.

11. The method of claim 5, wherein the at least one electrode is located longitudinally along a distal aspect of the lead.

12. The method of claim 5, wherein the electrode is positioned with a directional bias in around the lead diameter.

13. The method of claim 5, where the electrode is directed toward the autonomic nerve.

14. The method of claim 5, wherein the lead comprises a guidewire lumen.

15. The method of claim 14, further comprises
inserting the guidewire lumen at least one centimeter into the vertebral vein; and
advancing the lead over the guidewire.

16. A system for treating a subject with an electronic device for neuromodulation, comprising:
a lead configured to be inserted into a vertebral vein of the subject via a subclavian vein, an axillary vein, or a combination thereof;
at least one electrode coupled to the lead, wherein the at least one electrode is configured to be placed in the vertebral vein adjacent to an autonomic nerve, wherein the at least one electrode is coupled to the lead; and
a pulse generator coupled to the lead, wherein the pulse generator is configured to supply an electrical stimulation energy to the autonomic nerve through the lead.

17. The system of claim 16, wherein the system is for guiding a cardiac ablation procedure.

18. The system of claim 16, wherein a frequency of the electrical stimulation energy ranges from about 0.9 Hz to about 10 kHz.

19. The system of claim 17, wherein the amplitude of electrical stimulation ranges from about 2-20 mA.

20. The system of claim 16, wherein the electronic device is implantable.

21. The system of claim 20, wherein a diameter of the lead ranges from about 0.6 mm to about 2.5 mm.

22. The system of claim 20, wherein the lead comprises a tapered distal tip with a bend for positioning in the vertebral vein and ensuring contact with the autonomic nerve.

23. The system of claim 20, wherein the lead comprises up to about 20 electrodes.

24. The system of claim 20, where the lead is configured to exert a bias force to retain a distal top of the lead substantially immobile.

25. The system of claim 20, wherein the lead is configured to detect an electrical energy corresponding to a ventricular contraction, a thoracic impedance, or a combination thereof.

26. The system of claim 20, wherein the pulse generator is configured to detect ventricular arrhythmias based on a rate of a ventricular activity and a direction of activation.

27. The system of claim 20, wherein the pulse generator is a battery powered pulse generator.
